(19) United States
(12) Patent Application Publication
(10) Pub. No.: US 2003/0076028 A1
(43) Pub. Date: Apr. 24, 2003

(54) Method of activating photocatalysis, photocatalytic discharge tube, and device using the same

(76) Inventors: Manabu Nieda, Tokyo (JP); Yoriyuki Nieda, Kamakura-City (JP)

Correspondence Address: Kazuyuki Nieda, DTS 5171 Clareton Drive, Agoura Hills, CA, 91301, US

(21) Appl. No.: 10/267,315

(22) Filed: Oct. 9, 2002

(30) Foreign Application Priority Data
Oct. 19, 2001 (JP) JP2001-322071

(51) Int. Cl. H01J 1/62; H01J 63/04

(52) U.S. Cl. 313/485

(57) ABSTRACT

A discharge tube comprises a visible light region phosphor and a photocatalysis region phosphor that emits near-ultraviolet light. The discharge tube radiates visible light and near infrared light so as to promote plant growth by way of photocatalysis.

### BACKGROUND OF THE INVENTION

1. Field of the Invention

The present invention relates to a method of activating photocatalysis, to a photocatalytic discharge tube, and to a device which uses these principles.

2. Description of the Related Art

Fluorescent lamps and incandescent lamps are commonly used as light sources for plant cultivation. These light sources emit visible light which can be used in photosynthesis by the plant.

In addition to visible light, near-ultraviolet, and infrared can be used by plants in photosynthesis, but at the present time almost no use is made of the near-ultraviolet region and the infrared region in light sources for plant cultivation.

The absorption characteristics of light required for plant cultivation, which is to say photosynthesis, are those of blue light (400 to 500 nm wavelength) and red light (600 to 700 nm). The intermediate green to yellow light (500 to 600 nm) has substantially no photosynthesis effect.

If near-ultraviolet (300 to 400 nm) and infrared (700 to 800 nm) are added to this blue and red light, the photosynthesis is greatly promoted, and plants grow nearly twice as fast.

The glass tubes of currently used fluorescent lamps reach temperatures of 150° C. at the end of their working life, which is a problem in that drops of water from sprinklers, etc., may cause these fluorescent lamps to shatter, resulting in pieces of glass falling on the plants and rendering them worthless. Accordingly, fluorescent lamps had to be installed in high positions, such as on the ceiling. Thus, as a matter of course, the radiant energy of the incident light was reduced by at least a factor of ten.

Fluorescent lamps are made of soft glass, the physical characteristics of which do not allow for the transmission of light having wavelengths below 280 nm. Consequently, such light is not radiated to the exterior of the glass tube. Thus 253.7 nm ultraviolet light, which is generated within the fluorescent lamp, cannot be utilized for plant growth, which is to say, for photosynthesis. Furthermore, 368 nm near-ultraviolet is necessary for photocatalysis, but this ultraviolet light is transmitted by soft glass.

There are a number of problems associated with plant cultivation that can be solved by:

1) promoting photosynthesis in plants, so as to accelerate growth, and advance harvests, so as to increase market value;

2) ensuring growth and harvests by making these independent of the weather;

3) saving time during harvest and shipping by degrading, and thus removing, agricultural chemicals such as pesticides and antibacterial agents; and

4) reducing such production maintenance costs as those associated with the life of grow lamps and power consumption.

### SUMMARY OF THE INVENTION

A primary object of the present invention is to provide a photocatalytic discharge tube which can solve these various problems. Furthermore, the present invention provides a method of activating photocatalysis and a device which uses the photocatalytic discharge tube.

According to one aspect of the present invention, a photocatalytic discharge tube is provided which generates visible light and near-ultraviolet light.

According to another aspect of the present invention, a photocatalytic discharge tube is provided wherein this discharge tube further generates infrared light.

Other discharge tubes, according to the present invention, are set forth in the dependant claims.

A further aspect of the present invention provides a method of using this discharge tube to activate photocatalysis, wherein liquid or powdered titanium oxide is mixed with liquid or powdered antibacterial agents or fertilizer, and this mixture is irradiated with light from the discharge tube so as to activate photocatalysis.

Yet another aspect of the present invention provides a method of using this discharge tube to activate photocatalysis, wherein a coating of titanium oxide is formed on surfaces, such as the ceiling, walls, floor of a tunnel or building, or on the interior of a refrigerator, and this is irradiated with light from the discharge tube so as to activate photocatalysis.

A still further aspect of the present invention provides a water purifier comprising: a water tank having a water inlet and a water outlet; a lid which covers the water tank; and a discharge tube which is suspended in this water tank from this lid. This discharge tube is provided with a phosphor in the photocatalytic range which emits near-ultraviolet light, and a titanium oxide coating is provided on the inner walls of this water tank.

### BRIEF DESCRIPTION OF THE DRAWINGS

FIG. 1 is a partial sectional view of a fluorescent lamp according to the present invention, illustrating the relationship between this and the facility in which it is installed.

FIG. 2 is a graph illustrating the characteristics of the fluorescent lamp in FIG. 1.

FIG. 3 is a partial sectional view of a fluorescent lamp in another mode of embodiment of the present invention.

FIG. 4 is a graph illustrating the characteristics of the fluorescent lamp in FIG. 3.

FIG. 5 is a partially cut away overall perspective view of a fluorescent lamp in yet another mode of embodiment of the present invention.

FIG. 6 is a partially cut away overall perspective view of a partially modified fluorescent lamp from FIG. 5.

FIG. 7 is a partially cut away overall perspective view of the fluorescent lamp in FIG. 3.

FIG. 8 is a partially cut away overall perspective view of a variant fluorescent lamp wherein the titanium oxide coating is not applied to a portion of the glass tube.

FIG. 9 is a sectional view according to the line VIIII-VIIII in FIG. 8.

FIG. 10 is a sectional view of a fluorescent lamp having terminals for the purpose of extending the life thereof.

FIG. 11 is a sectional view of a water purifier using the discharge tube of the present invention.

### DETAILED DESCRIPTION OF THE INVENTION

Firstly, while various types of photocatalytic discharge tubes are available, including fluorescent lamps, mercury lamps, sodium lamps, etc., an example of a fluorescent lamp will be described hereinafter. It will, however, be appreciated that the discharge tube of the present invention is not limited to fluorescent lamps.

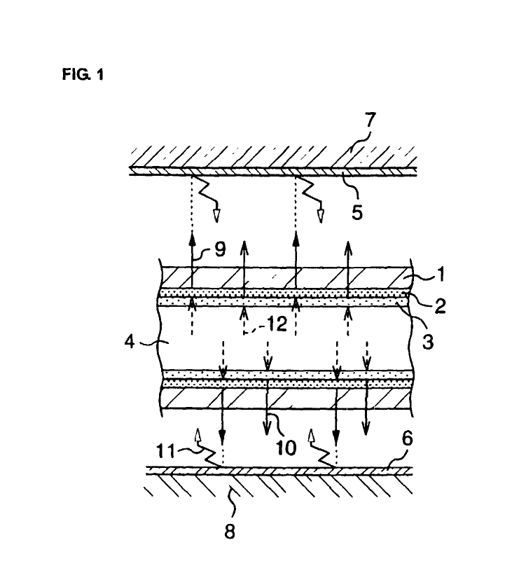

FIG. 1 is a partial sectional view of a fluorescent lamp of the present invention, illustrating the relationship between this and a facility in which it is installed. A near-ultraviolet emitting phosphor layer 2 and a visible light emitting phosphor layer 3 are successively provided on the inner face of a glass tube 1; an inert gas 4, such as mercury or argon, is sealed therein. A ceiling 7 of the facility is located above the fluorescent lamp, and a titanium oxide coating 5 is provided thereon; a floor 8 of the facility is located below the fluorescent lamp, and a coating 6 of titanium oxide is provided thereon.

The fluorescent lamp in FIG. 1 is used for plant cultivation and is provided with terminals at either end thereof, which are not shown in the drawing. The near-ultraviolet emitting phosphor layer 2 is market available. For example [BaSi₂O₅:Pb or (CaZn)₃(PO₄)₂:Ti⁺] or Ca₃(PO₄)₂:TlGd, etc., can be applied. A visible light emitting phosphor layer 3 is formed as a coating of, for example, [3Ca₃(PO₄)₂.Ca(FCl)₂:Sb3⁺, Mn²⁺] on the surface of this coating and, in addition to visible light, infrared light can easily be generated by using three fluorescent materials that emit differing wavelengths, including an infrared phosphor, such as the Toshiba phosphor SPD-120 or (Sr, Mg, Ba)₃(PO₄)₂:Sn.

In FIG. 1, if electric power is applied to the fluorescent lamp, ultraviolet light 12 with a wavelength of 253.7 nm, indicated by the arrows, is generated by the mercury vapor at the interior. This excites the visible light emitting phosphor layer 3, which emits visible light 10, indicated by the arrows, and this is radiated to the exterior of the glass tube 1. Furthermore, the successively provided near-ultraviolet phosphor layer 2 is excited and emits near-ultraviolet light 9 with a wavelength of 368 nm, indicated by the arrows, which is radiated to the exterior of the glass tube 1. At this point, some of the near-ultraviolet light 9 is radiated to the interior of the glass tube 1 and excites the visible light emitting fluorescent layer 2 so as to increase the brightness.

Ultraviolet light with a wavelength of 253.7 nm is almost entirely absorbed by soft glass, and only a very small amount, not exceeding 0.5% of the energy consumption, is radiated to the exterior of the glass tube. However, as soft glass transmits light with a wavelength of 280 nm or greater, the near-ultraviolet light with a wavelength of 368 nm is readily transmitted. Furthermore, the titanium oxide coatings 5 and 6 on the ceiling 7 and the floor 8, which are particularly irradiated by the near-ultraviolet, are activated and, photocatalysis occurs, as symbolically indicated by the arrows 11.

Conventional fluorescent lamps generate only ultraviolet light with a wavelength of 253.7 nm, but with the fluorescent lamp of the present invention, as a result of the combined excitation effect of the 253.7 nm ultraviolet light and the reflected 368 nm near-ultraviolet light, with a 40 W lamp, 2500 lm (lumen) are produced, as compared to 1300 lm with a conventional lamp.

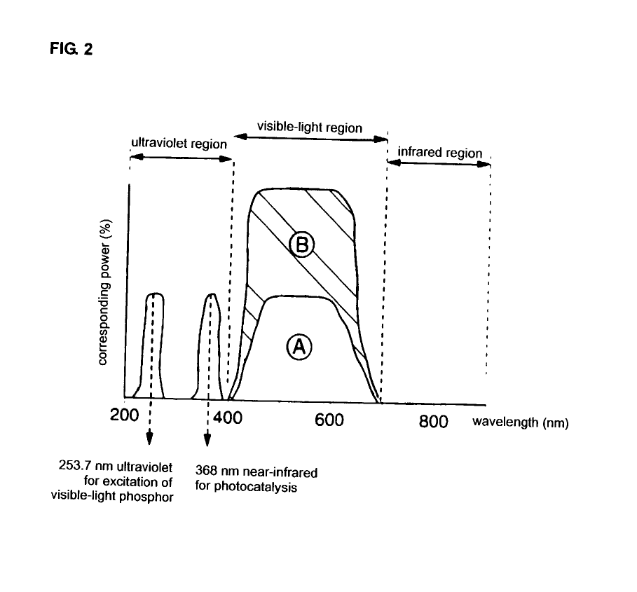

FIG. 2 shows the characteristics of the fluorescent lamp in FIG. 1. An amount of light in the visible light region indicated by A is produced by the 253.7 nm ultraviolet, while an amount of light in the visible light region indicated by B is produced by the 368 nm near-ultraviolet.

Next, the photocatalysis 11 illustrated in FIG. 1 is described in detail. If the coatings 5 and 6 of titanium oxide (TiO₂) are irradiated with 360 nm near-ultraviolet light, the oxygen and water at the surfaces thereof react, and a redox reaction occurs, producing hydrogen peroxide (H₂O₂) and hydroxyl radicals, whereby bacteria are oxidatively degraded. This further reacts with organic matter (fertilizers, etc.) to form carbon dioxide gas and water, whereby organic fertilizers and the like are deodorized approximately 130 times as effectively as with activated carbon, which keeps the air in the cultivation rooms extremely fresh and comfortable. As shown in FIG. 1, titanium oxide coatings 5 and 6 are formed on the ceiling 7 and the floor 8 and irradiated by the fluorescent lamp of the present invention, whereby dangerous places, such as tunnels, where operations are difficult, are kept clean and are deodorized and sterilized, while microorganisms are degraded, allowing for great reductions in upkeep and maintenance costs.

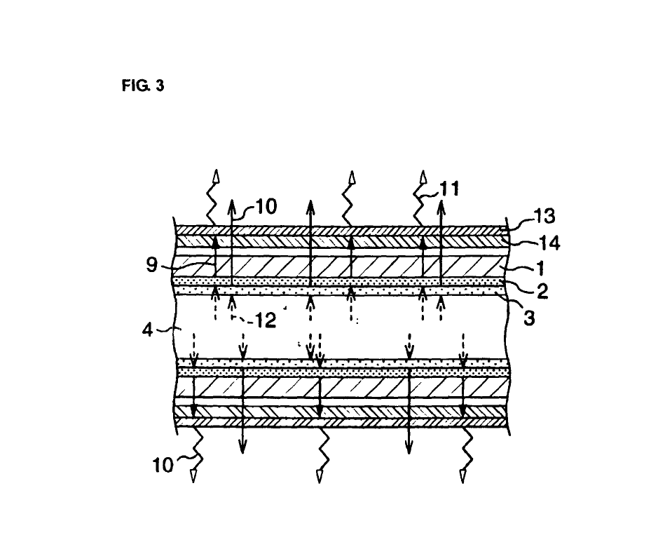

FIG. 3 is a partial sectional view of a fluorescent lamp in another embodiment of the present invention. Here, the same reference numerals as in FIG. 1 indicate the same parts. A transparent Teflon™ tube 14 is provided around the glass tube 1 of this fluorescent lamp, and a transparent coating 13 of titanium oxide is formed at the outer surface thereof. At the end of its working life, the glass tube of the fluorescent lamp reaches temperatures of 150° C. and can easily be shattered by drops of water as described above. In order to prevent this, the fluorescent lamp in FIG. 3 is one wherein the entire outer circumference of the glass tube 1 of the fluorescent lamp in FIG. 1 is covered by a Teflon™ tube 14, and a titanium oxide coating 13 is formed at the outer circumference thereof. The Teflon™ tube 14 is colorless and transparent, and visible light and 368 nm near-ultraviolet light, which are transmitted by the glass tube 1, are transmitted by the Teflon™ tube 14, reach the titanium oxide coating, and excite this. This brings about photocatalysis and, as a result of a redox reaction which occurs at the surface thereof, degradation of contaminants, deodorizing, sterilization, and an anti-soiling effect are efficiently provided.

Furthermore, as a variant, in place of the Teflon™ tube 14, a transparent heat shrink plastic film may be affixed to the entire outer face of the glass tube, and a titanium oxide coating may be formed on the surface thereof, which produces the same effect.

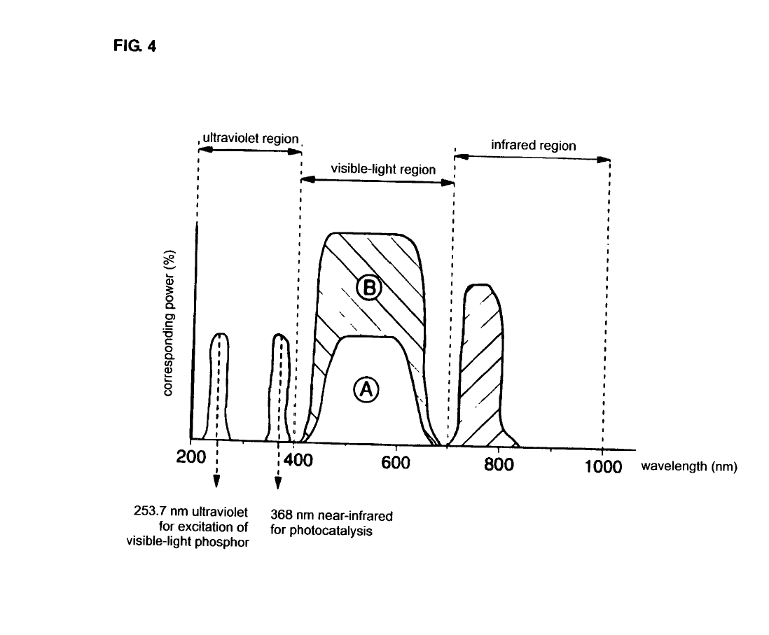

FIG. 4 is a graph illustrating the characteristics of the fluorescent lamp in FIG. 3. If a visible-light emitting phosphor is combined with an infrared phosphor, the ultraviolet region and infrared region are superimposed on the visible light region, as shown in FIG. 2, and the visible light is increased by 1.7 to 2.0 times, as indicated by B in FIG. 4. Thus, as the total of B, the ultraviolet region and the infrared region is used for photosynthesis. This has been confirmed to be more than twice as effective as conventional fluorescent lamps.

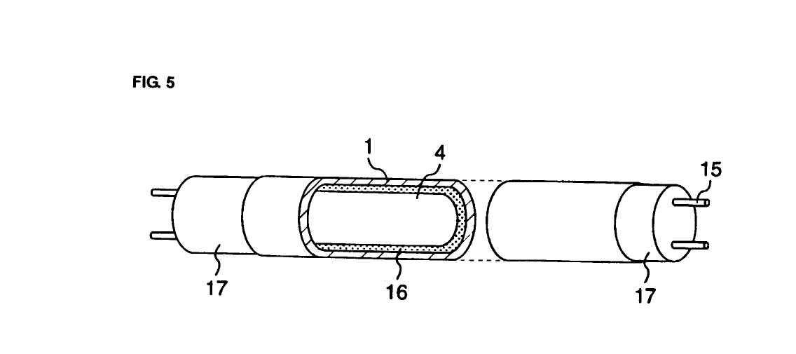

FIG. 5 is a partially cut away overall perspective view of a fluorescent lamp according to another embodiment of the present invention, illustrating connectors 17 and pins 15. This fluorescent lamp does not have a two-layer coating of the type shown in FIG. 1; rather a coating 16 is formed wherein visible-light, infrared, and near-ultraviolet phosphors are combined in one layer.

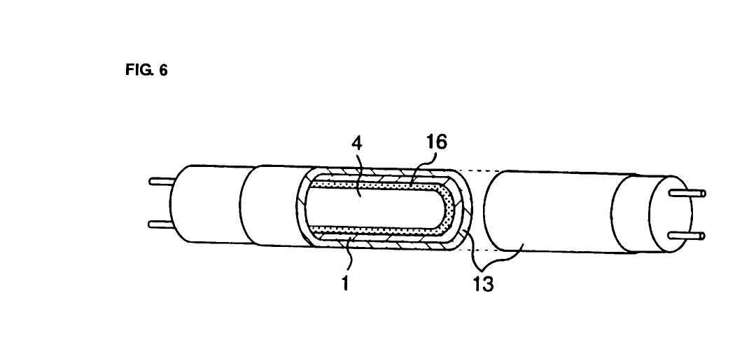

FIG. 6 is a partially cut away overall perspective view of a variant wherein a transparent titanium oxide coating 13 is provided over the entire outer face of the glass tube 1 of the fluorescent lamp in FIG. 5.

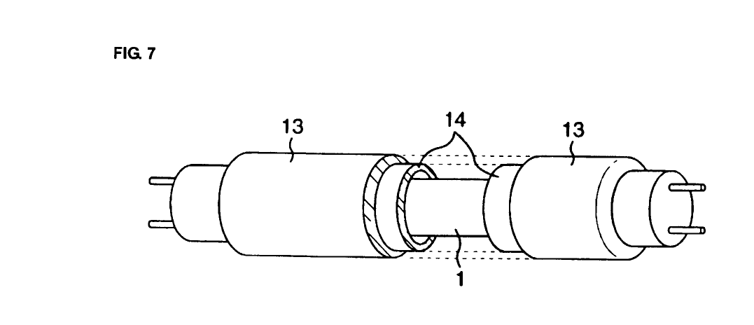

FIG. 7 is an overall perspective view of the fluorescent lamp in FIG. 3. Photosynthesis and photocatalysis can be effectuated at maximum efficiency if this is used for illumination at 20 to 40 cm above the soil surface.

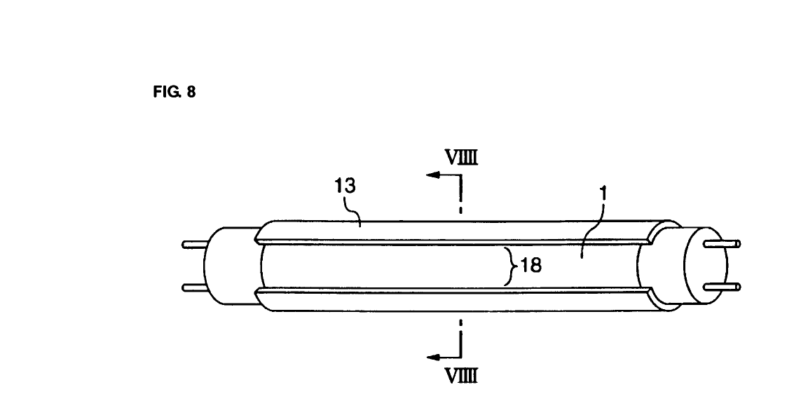

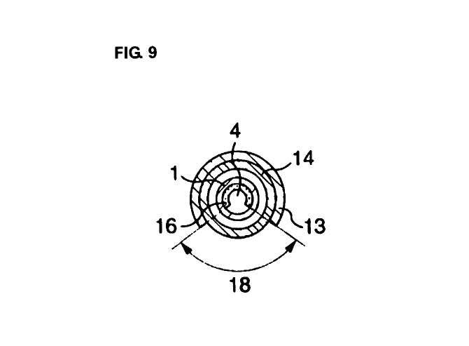

In FIG. 8, a titanium oxide coating 13 and a phosphor layer 16 (see FIG. 9) on the outer face of the tube are not applied over the entire surface as in FIG. 6 and FIG. 7; rather, in this example, an aperture 18 at which these are not applied is provided in the lengthwise direction of the fluorescent lamp. This can be achieved by coating the entire circumference and then removing a portion of this.

FIG. 9 is a sectional view according to line VIIII-VIIII in FIG. 8; a single coating layer 16 is formed on the fluorescent lamp in FIG. 8, as described in relation to FIG. 5. The phosphor layer 16 allows the phosphor to be applied as a thick coating, whereby the light from the aperture is made even brighter. Furthermore, various modes are possible in addition to the mode described above wherein a titanium oxide coating and phosphor layers are partially applied.

A fluorescent lamp, such as shown in FIG. 8, can be used to brighten lighting in passageways, such as in tunnels. Furthermore, this is effective in maximizing the photocatalytic effect on other walls or ceilings by increasing the thickness of the titanium oxide layer regardless of the transparency thereof.

In the foregoing description, a relationship between titanium oxide (TiO₂) and 368 nm near-ultraviolet light which excites this is described, but in addition to TlO₂, tungsten trioxide (WO₃) or the like can be used as the photocatalysis. Accordingly, in the present invention, the substance which activates photocatalysis is not limited to titanium oxide. Furthermore, photocatalysis can be activated with the substance which activates photocatalysis applied to the surface of the fluorescent lamp, the surface of a fitting in which the fluorescent lamp is mounted, or the surface of an object which is illuminated by the fluorescent lamp, and therefore, this can be applied to a broad range of places.

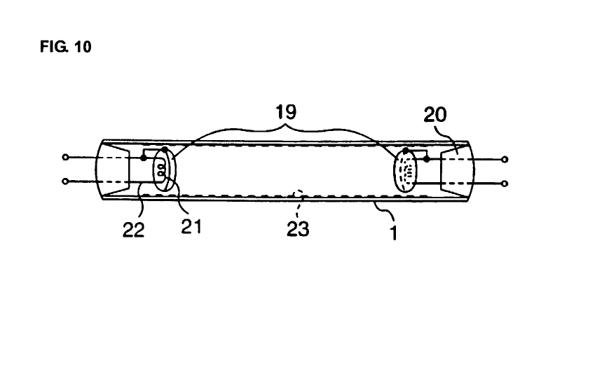

The terminal structure for extending the life of the fluorescent lamp of the present invention is shown in FIG. 10 in a sectional view of the fluorescent lamp. Dome-shaped or cylindrical metal electrodes 19 are formed as enclosures which surround substantially all of filament coils 21 (center terminals), which are supported by stems 20, without contacting the filament coils 21; an edge of each metal electrode 19 is connected to one of leads 22 and supported thereby. A phosphor layer or a plurality of layers 23, according to the various examples described above, is provided on the inner face of the glass tube 1. As a result of this construction, it is possible to extend the life of the fluorescent lamp by 3 to 5 times that of conventional fluorescent lamps, which is highly economical.

The foregoing description is primarily a description of photocatalytic discharge tubes for plant cultivation, but there is also a photo-oxidation breakdown reaction wherein photocatalytic titanium oxide absorbs 368 nm near-ultraviolet light, and substances are degraded; in other words, wherein organic substances are broken down into carbon dioxide and water. This provides a self-cleaning effect wherein contaminants, chemicals, and microorganisms are degraded. There is also a hydrophilic reaction which allows for easy removal of soiling. As these two reactions are optimal for environmental clean-up and can be applied to water purifiers, a water purifier using a photocatalytic discharge tube is described hereinafter.

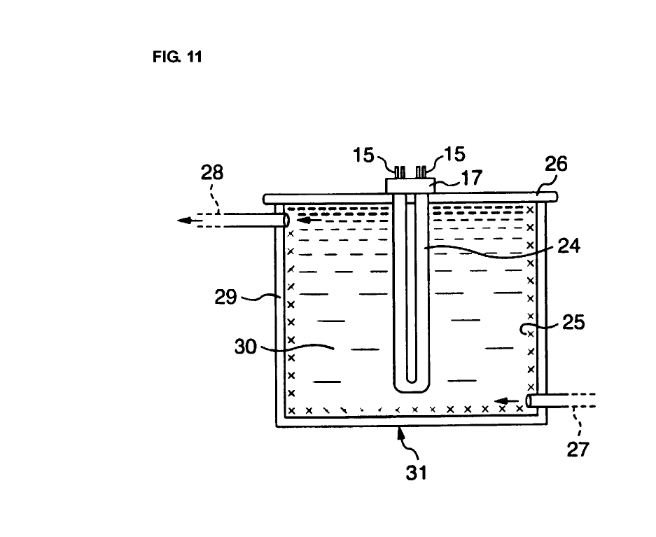

FIG. 11 is a sectional view of a water purifier 31 of the present invention. As there is no need for the fluorescent lamp used in the water purifier to generate visible light, the visible light emitting phosphor layer shown in FIG. 1 and FIG. 3 is not necessary, thus a discharge tube 24 is used wherein only a near-ultraviolet light emitting phosphor layer is provided on the inner face of the glass tubes. If, instead of soft glass, quartz glass or Pyrex™ glass is used for the glass tube, 253.7 nm ultraviolet light is radiated to the exterior of the glass, allowing for utilization of the sterilization effect.

This water purifier 31 comprises a water tank 29, having an inner surface on which a titanium oxide coating 25 is formed, and a water tank lid 26. The water tank 29 is provided with an inlet 27, through which water is introduced, and an outlet 28, through which water is discharged. A U-shaped discharge tube 24 comprises a glass tube of Pyrex™, etc., as described above, and is suspended in the water 30 within the water tank 29 from the lid 26. The discharge tube 24 emits 253.7 nm ultraviolet light, which has a sterilizing effect, and 368 nm near-ultraviolet light, which activates photocatalysis in the titanium oxide coating 25, which has a self-cleaning effect consisting of a photo-oxidization reaction and a hydrophilic reaction. As a result of the combined effects of this self-cleaning action and the sterilizing/antibacterial action of the 253.7 nm ultraviolet light described above, the water 30 in the water tank 29 is purified, which is 100 times as effective as well-known silver-based antibacterial agents.

By virtue of the photocatalytic discharge tube of the present invention, the following effects are achieved.

1) As a result of the combined effect of the near-ultraviolet light having a wavelength of 368 nm for photocatalysis and the ultraviolet light having a wavelength of 253.7 nm generated by the mercury vapor, as compared to the luminous flux of 1300 lm (lumen) produced by a conventional 40 W discharge tube, it is possible to produce a luminous flux of 2500 lumen.

2) As a result of that described in 1) above, the plant photosynthesis is promoted, ensuring and accelerating both plant growth and harvest, which shortens working time.

3) Using near-ultraviolet light having a wavelength of 368 nm for photosynthesis, which was completely unused in the past, renders the leaves of the plants thick and sturdy, allowing a modified morphology, with short plants having limited succulent growth, whereby product value is increased.

4) Photocatalysis degrades toxins created by microbes which grow on the plant and in the soil as well as both the dead bodies of insects killed by antibacterial agents and insecticides in a self-cleaning effect.

5) Photocatalysis kills, and prevents the proliferation of, microbes due to a photocatalytic breakdown reaction wherein organic substances in fertilizer are degraded, which is several tens of times more effective than silver-based antibacterial agents.

6) Due to the hydrophilic effect of photocatalysis, soiling of plant leaves is prevented, and due to an anti-contamination effect whereby indoor airborne odors and contamination are prevented, interiors can be kept fresh, and a good environment can be maintained.

7) Photocatalysis can prevent the growth of moss on plant leaves and soil.

8) Microorganisms which grow on floors, walls, ceilings, etc., in hospitals can be killed and their proliferation prevented.

9) Power consumption is exactly the same as with conventional devices.

10) Lighting equipment (grow lamps, high-frequency lamps, etc.) and fittings (mountings, reflectors, etc.) used with conventional discharge lamps can be used without modification.

11) If titanium oxide is used, there is no change whatsoever to the substance itself upon absorbing light, and therefore, a (semi-permanent, low-cost) discharge tube can be provided which has an extremely long life and is economical.

By using the discharge tube described above in microwave ovens or dish dryers, in addition to the sterilizing effect and the deodorizing effect within the chamber, bacteria which is not killed by the heat can be killed.

Furthermore, if this is used in a washing machine, soiling can be degraded without detergent before washing. Moreover, following discharge of the water, disinfection of saprophytic bacteria, etc., and deodorizing is possible.

In addition, if this is used in a refrigerator, it is possible to adjust the ripening of foods in the refrigerator as a result of photocatalysis. Moreover, this is effective in eradicating saprophytic bacteria which tend to proliferate when bag-packaged food is defrosted.

We claim:

1. A photocatalytic discharge tube that generates infrared light and near-ultraviolet light in the region effective for photocatalysis.

2. A photocatalytic discharge tube that generates visible light and near-ultraviolet light in the region effective for photocatalysis.

3. The photocatalytic discharge tube of claim 2, wherein said discharge tube further generates infrared light.

4. The photocatalytic discharge tube of any one of claims 1 to 3, wherein this comprises a glass tube, and a translucent titanium oxide film is provided on the entire outer face of said glass tube.

5. The photocatalytic discharge tube of any one of claims 1 to 3, wherein this comprises a glass tube and a transparent heat-shrink plastic film, or a Teflon™ tube is affixed to the entire outer face of said glass tube, and a translucent titanium oxide film is further provided on the outer surface thereof.

6. The photocatalytic discharge tube of any one of claims 1 to 3, wherein this comprises a glass tube and a phosphor that emits said near-ultraviolet light in the region effective for photocatalysis is provided on said glass tube.

7. The photocatalytic discharge tube of claim 6, wherein said phosphor forms an aperture so as to allow light radiated from said glass tube to pass directly therethrough.

8. The photocatalytic discharge tube of any one of claims 1 to 3, wherein this comprises a glass tube, and a plurality of phosphors which emit said types of light are provided as separate layers on the interior of said glass tube.

9. The photocatalytic discharge tube of any one of claims 1 to 3, wherein this comprises a glass tube, and a plurality of phosphors which emit said types of light are provided as one combined layer on the interior of said glass tube.

10. The photocatalytic discharge tube of any one of claims 1 to 9, wherein said discharge tube comprises a terminal device comprising a metal electrode that at least partially surrounds a center electrode without contacting it, and wherein one edge of said metal electrode is connected to one of two leads, which are connected to both edges of said center electrode, and is supported thereby.

11. The photocatalytic discharge tube of any one of claims 1 to 10, wherein said discharge tube is a fluorescent lamp.

12. A method of activating photocatalysis using the discharge tube of any one of claims 1 to 11, wherein liquid or powdered titanium oxide is mixed with liquid or powdered antibacterial agents or fertilizer, and this mixture is irradiated with light from said discharge tube so as to activate photocatalysis.

13. A method of activating photocatalysis using the discharge tube of any one of claims 1 to 11, wherein a titanium oxide solution or a titanium oxide powder is irradiated with light from said discharge tube so as to activate photocatalysis.

14. A method of activating photocatalysis using the discharge tube of any one of claims 1 to 11, wherein a titanium oxide coating is applied to an exposed wall face, and said titanium oxide coating is irradiated with light from said discharge tube so as to activate photocatalysis.

15. A water purifier comprising a water tank having a water inlet and a water outlet, a lid which covers the water tank, a discharge tube which is suspended in this water tank from this lid, said discharge tube being provided with a phosphor in the photocatalytic range which emits near-ultraviolet light, and a titanium oxide coating being provided on the inner walls of this water tank.
